(19) United States
(12) Patent Application Publication
(10) Pub. No.: US 2023/0326611 A1
(43) Pub. Date: Oct. 12, 2023

(54) SYSTEMS AND METHODS FOR CONTROLLING ILLNESS RISK INFORMATION

(71) Applicant: Temperature SafeNet, Inc., Washington, DC (US)

(72) Inventor: Jerrod Edward Moton, JR., Washington, DC (US)

(73) Assignee: Temperature SafeNet, Inc., Washington, DC (US)

(21) Appl. No.: 17/714,759

(22) Filed: Apr. 6, 2022

(51) Int. Cl. G16H 50/80; G16H 10/20; G16H 50/30; H04L 51/046

(52) U.S. Cl. CPC G16H 50/80; H04L 51/046; G16H 50/30; G16H 10/20

(57) ABSTRACT

A system for controlling notifications relating to illness information includes a processor and memory. The processor is configured to set first criteria for (1) vital information, (2) questionnaire response information, (3) vaccination information, and (4) illness test result information, the first criteria relating to a first illness, receive first data relating to an illness risk of a first user, the first data including (1) vital information, (2) questionnaire response information, (3) vaccination information, and (4) illness test result information, of the first user, and determine, based on the first criteria and the first data, whether the first user has an illness risk. In response to determining that the first user has an illness risk, the processor is configured to send to the first user an illness risk notification.

### TECHNICAL FIELD

The present disclosure relates to systems and methods for controlling illness risk information and more particularly to systems and methods for receiving data relating to an illness risk of a user and controlling notification relating to the illness risk.

### BACKGROUND

Under a virus pandemic situation, like COVID-19, where there are ongoing needs for antiviral treatments for new variants of a virus, preventive measures such as monitoring and self-isolation for people who suspect they are infected are strongly recommended. Ongoing monitoring in a workplace can be performed by developing and implementing procedures to check for signs and symptoms of employees daily upon arrival and to encourage anyone who is sick to stay home, and to monitor employee absences. Such ongoing monitoring also can be performed at home. Under this situation, improvements remain desired in a system for managing and controlling illness risk information (e.g., information relating to a person's risk of developing a particular illness) and determining measures based on the illness risk information.

### SUMMARY

Implementations of the present disclosure relate to a system and a method for controlling illness risk information and more particularly to systems and methods for receiving data relating to an illness risk of a user and controlling notification relating to the illness risk.

In some implementations of the present disclosure, a system for controlling notifications relating to illness information may include a processor and memory. The processor may be configured to set first criteria for (1) vital information, (2) questionnaire response information, (3) vaccination information, and (4) illness test result information, the first criteria relating to a first illness. The processor may be configured to receive first data relating to an illness risk of a first user, the first data including (1) vital information, (2) questionnaire response information, (3) vaccination information, and (4) illness test result information, of the first user. The processor may be configured to determine, based on the first criteria and the first data, whether the first user has an illness risk. In response to determining that the first user has an illness risk, the processor may be configured to send to the first user an illness risk notification.

In some implementations of the present disclosure, a method for controlling notifications relating to illness information may include setting, by a processor, first criteria for (1) vital information, (2) questionnaire response information, (3) vaccination information, and (4) illness test result information, the first criteria relating to a first illness. The method may include receiving, by the processor, first data relating to an illness risk of a first user, the first data including (1) vital information, (2) questionnaire response information, (3) vaccination information, and (4) illness test result information, of the first user. The method may include determining, by the processor based on the first criteria and the first data, whether the first user has an illness risk. The method may include in response to determining that the first user has an illness risk, sending, by the processor, to the first user an illness risk notification.

### BRIEF DESCRIPTION OF THE DRAWINGS

These and other aspects and features of the present implementations will become apparent to those ordinarily skilled in the art upon review of the following description of specific implementations in conjunction with the accompanying figures, wherein:

FIG. 1 is a block diagram illustrating an example of a system environment for an illness information control system according to some implementations.

FIG. 2 is a block diagram illustrating an example of a computing system according to some implementations.

FIG. 3A to FIG. 3F are example user interfaces for displaying illness information on a mobile device according to some implementations.

FIG. 4 is an example user interface for displaying illness information for an enterprise environment according to some implementations.

FIG. 5A to FIG. 5D are example user interfaces for receiving or inputting different categories of illness information from users according to some implementations.

FIG. 6A and FIG. 6B are example user interfaces for displaying different categories of illness information according to some implementations.

FIG. 7A and FIG. 7B are example user interfaces for adding and editing compliance criteria on different categories of illness information according to some implementations.

FIG. 8A to FIG. 8D are example user interfaces for displaying illness information based on one or more compliances according to some implementations.

FIG. 9A to FIG. 9C are example user interfaces for controlling notifications relating to illness information according to some implementations.

FIG. 10 is a flowchart illustrating an example methodology for controlling notifications relating to illness information according to some implementations.

### DETAILED DESCRIPTION

According to certain aspects, implementations in the present disclosure relate to a system and a method for controlling illness risk information and more particularly to systems and methods for receiving data relating to an illness risk of a user and controlling notification relating to the illness risk.

Under a virus pandemic situation, like COVID-19, where there are ongoing needs for antiviral treatments for new variants of a virus, ongoing monitoring in a workplace can be performed by developing and implementing procedures to check for signs and symptoms of employees frequently (e.g., daily upon arrival). For example, the Executive Office of the White House has mandated agencies deploy the following: “Onsite federal employees, onsite contractor employees, and visitors who need to report to the workplace must complete symptom screening before they report each day.” Such ongoing monitoring also can be performed at home. As vaccines or tests for a virus become available, improvements remain desired in a system for managing and controlling information relating to vaccine administration and/or test results (as illness risk information) and determining measures (e.g., quarantine) based on the illness risk information.

To solve the above-noted problems, according to certain aspects, an illness information control system can provide an enterprise environment the ability to offer a safe environment for employees and customers. The system can perform screening, surveillance and/or monitoring to prevent a particular illness (e.g., COVID-19) based on illness risk information. For example, the illness risk information may include vital information (e.g., body temperature), vaccination information (e.g., information from a vaccination card certificate using optical character recognition (OCR)), symptom information (e.g., information from a symptom questionnaire), test information (e.g., results of COVID-19 tests), of a person (e.g., employees, customers, visitors). In some implementations, the system may generate a barcode (e.g., quick response (QR) code) to identify such illness risk information or data (e.g., information relating to body temperature, symptoms, vaccine administration, test result) of a particular person/patient. The barcode can be scanned (e.g., scanned by a camera of a mobile device) for verification of illness risk without revealing detailed patient data.

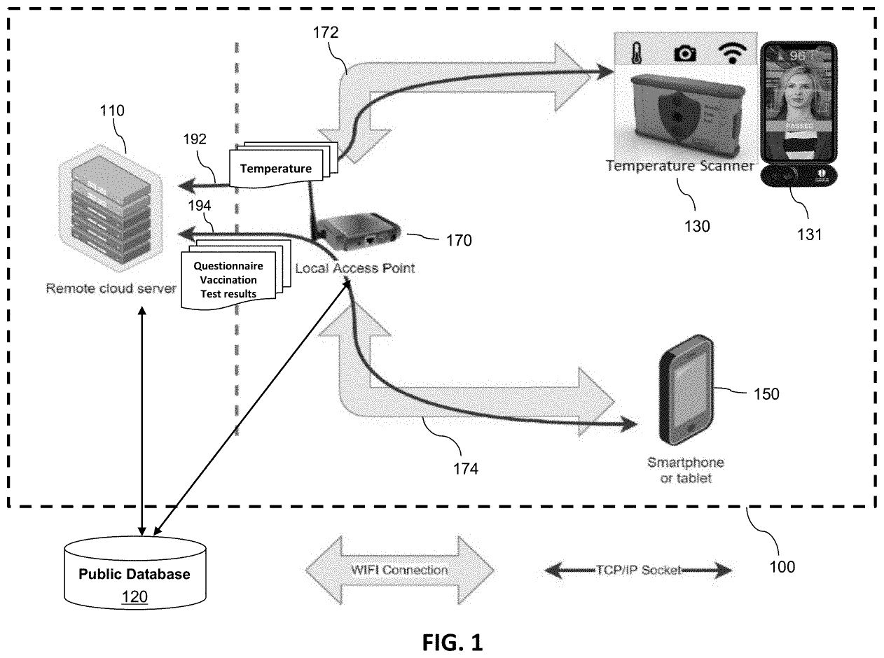

In some implementations, an illness information control system may allow illness risk information or data of employees or visitors to be shared with an employer. In some implementations, the system may allow users to share their illness risk information or data (with employer/company/organization etc.) from anywhere (e.g., while working home) using their mobile devices. In some implementations, the illness risk information or data may be uploaded to and integrated to an enterprise system (e.g., an illness information control system 100 in FIG. 1). The system may combine and use different categories of the illness risk information or data (e.g., four different categories—information relating to body temperature, symptoms, vaccine administration, test results). In some implementations, the system may integrate (or have access to) a public database (e.g., state or local vaccination database; a public database 120 in FIG. 1) to provide the illness risk information or data (e.g., vaccine administration data).

In some implementations, in an enterprise environment, an illness information control system may perform functions including (1) collecting, managing, and/or displaying temperatures of employees/visitors, (2) locating, reading, accessing, and/or displaying a digital vaccination certification of an employee/visitor, (3) collecting or reading or capturing (by OCR), managing, converting (to a digital document), and/or displaying a vaccination card of an employee/visitor, (4) creating, collecting, managing, and/or displaying a symptom questionnaire or symptom checklist, (5) locating, reading, accessing, and/or displaying a digital test result (of a particular illness, e.g., COVID-19 test result) of an employee/visitor, (6) collecting or reading or capturing (by OCR), managing, converting (to a digital document), and/or displaying a test result (of a particular illness, e.g., COVID-19 test result) of an employee/visitor, (7) setting and/or displaying different illness risk policies (or illness risk compliances) for different groups or departments, (8) tracking and/or displaying health metrics or illness risk metrics for each person (e.g., body temperature, symptoms, vaccine administration, test result), (9) managing and/or displaying human resources (HR) workflow (e.g., bulk employee management, managing wellness wallet or benefits) and/or (10) creating, editing, managing, displaying and/or controlling notification (or report) relating to wellness information or illness risk information of a person. These functions may be performed by a server device (e.g., server device 110 in FIG. 1) or a mobile device (e.g., mobile device 131, 150 in FIG. 1). In some implementations, these functions may be performed by one or more applications (e.g., “wellness app”, “scanner app”) in a mobile device. In some implementations, these functions may be performed by one or more applications (e.g., “administrative web application”) in a server device.

In some implementations, an illness information control system may allow a mobile device (e.g., through a “wellness” app) to perform a combination of at least one of (1) updating user data (e.g., email, phone), (2) configuring notification settings, (3) uploading documents (e.g., vaccination cards or test results) and survey answers (e.g., answers to a symptoms questionnaire), (4) generating a QR code, (5) generating a pre-filled document (e.g., pre-filled symptoms questionnaire) and modifying the document, (6) displaying in-app reports of health metrics (or illness risk metrics) including, for example, body temperature, symptoms, vaccine administration, test result, (7) controlling or sending reminders and notifications in-app or via text to other family members, (8) capturing or collecting user data including, for example, ID number, name, date of birth (DOB), gender, email, phone number, emergency contact, location, body temperature (and measurement date and time), documents (e.g., documents relating to vaccine administrations and/or test results), data relating to quarantine, wellness, and/or illness risk, (9) capturing documents using OCR, (10) scanning forms and save them, (11) generating and sharing virtual identity credentials (e.g., in the form of a QR code), (12) integrating with, or linking to, external wellness/health apps, (13) integrating with biometrics, (14) signing in with an external credential (e.g., using ID of a well-known app or ID of the mobile device), and/or (15) linking to external internet of things (IoT) devices (e.g., IoT devices in home).

In some implementations, an illness information control system may allow a mobile device or a server device (e.g., through a “scanning” app or a “scanning” web app) to perform a combination of at least one of (1) searching and filtering users and (health/illness) cases, (2) displaying and/or exporting detail and summaries of illness risk data, (3) displaying summary of user statistics, (4) managing visitor users and/or employee users, (5) scanning QR codes, (6) importing and storing illness risk data, (7) tracking health metrics (or illness risk metrics) including, for example, body temperature, symptoms, vaccine administration, test result, (8) updating user data (e.g., email, phone), (9) configuring notification settings, (10) generating a QR code, (11) generating a pre-filled document (e.g., pre-filled symptoms questionnaire) and modifying the document, (12) capturing or collecting user data including, for example, ID number, name, DOB, gender, email, phone number, business ID, location, time, visitor documents (e.g., documents relating to vaccine administrations and/or test results), and/or device ID, (13) capturing documents using OCR, (14) creating forms (e.g., creating a new symptom questionnaire), (15) importing or scanning forms, (16) integrating with biometrics, (17) performing age verification, (18) data sharing with government health agencies (e.g., through a public database 120 in FIG. 1), and/or (19) controlling or sending reminders and notifications in-app or via text to other family members.

In some implementations, an illness information control system may allow a mobile device or a server device (e.g., through a “wellness” app, a “scanning” app, or an “admin” web app) to perform a combination of at least one of (1) searching and filtering users and (health/illness) cases, (2) adding or removing a user to or from an organization, (3) viewing user details and listing user information, (4) bulk adding users to an organization, (5) viewing a total number of licenses owned by an organization, (6) creating and customizing settings of workforce compliances (e.g., illness risk compliances), (7) assigning (different) compliance settings to (different) user groups/departments, (8) assigning roles to users, (9) providing dynamic and customizable dashboards, (10) performing automate delivery of reports or notifications, (11) forecasting workforce wellness and quarantine metrics and providing/displaying historical reports of workforce wellness and quarantine metrics, (12) managing user accounts, (13) managing quarantine for in-person, remote, and hybrid situations, (14) managing visitor users, (15) updating user data (e.g., email, phone), (16) configuring notification settings, (17) tracking health metrics (or illness risk metrics) including, for example, body temperature, symptoms, vaccine administration, test result, (18) providing and/or displaying quarantine calendar for in-person, remote, or hybrid work environments, (19) uploading documents (e.g., vaccination cards or test results) and survey answers (e.g., answers to a symptoms questionnaire), (20) generating a QR code, (21) generating a pre-filled document (e.g., pre-filled symptoms questionnaire) and modifying the document, (22) capturing documents using OCR, (23) integrating with, or linking to, external wellness/health apps, (24) creating and populating e-sign forms, (25) integrating with biometrics, (26) supporting desktop and mobile single-sign-on (SSO), (26) capturing or collecting user data including, for example, ID number, name, DOB, gender, email, phone number, location, documents (e.g., documents relating to vaccine administrations and/or test results), device ID, message logs and/or wellness data (or illness risk data), (27) in-app messaging, (28) send email or short message service (SMS) to users as notification/report, and/or (29) controlling or sending reminders and notifications via SMS.

According to certain aspects, implementations in the present disclosure relate to a method and a system for controlling notifications relating to illness information. The system may include a processor and memory. The processor may be configured to set first criteria for (1) vital information, (2) questionnaire response information, (3) vaccination information, and (4) illness test result information, the first criteria relating to a first illness. The processor may be configured to receive first data relating to an illness risk of a first user, the first data including (1) vital information, (2) questionnaire response information, (3) vaccination information, and (4) illness test result information, of the first user. The processor may be configured to determine, based on the first criteria and the first data, whether the first user has an illness risk. In response to determining that the first user has an illness risk, the processor may be configured to send to the first user an illness risk notification.

In some implementations, the processor may be configured to determine, based on the first criteria and the first data, whether the first user is to be in quarantine. In response to determining that the first user is to be in quarantine, the processor may be configured to send to the first user an illness quarantine notification including time and duration of quarantine.

In some implementations, in setting the first criteria, the processor may be configured to set a criterion for each of (1) vital information, (2) questionnaire response information, (3) vaccination information, and (4) illness test result information. In setting the first criteria, the processor may be configured to set a group of users to which the first criteria are to be applied, wherein the group of users includes the first user. In determining whether the first user has an illness risk, the processor may be configured to compare each of the information (1) to (4) of the first data with the corresponding criterion, and determine, based on a result of the comparison, whether the first user has an illness risk.

In some implementations, the processor may be configured to set second criteria for (1) vital information, (2) questionnaire response information, (3) vaccination information, and (4) illness test result information, the second criteria relating to a second illness different from the first illness.

In some implementations, the vital information of the first user may include a measured body temperature of the first user. In some implementations, the questionnaire response information of the first user may include one or more responses to one or more questions relating to symptoms of the first illness. In some implementations, the vaccination information of the first user may include information relating to administration of one or more doses of vaccine of the first illness to the first user. In some implementations, the illness test result information of the first user may include information relating to administration of one or more tests of the first illness to the first user.

Various implementations in the present disclosure have one or more of the following advantages and benefits.

First, implementations in the present disclosure can allow a mobile device (e.g., through a mobile app) to control illness risk information of employees/visitors so that a user can share illness risk data with an enterprise server from anywhere (e.g., home). For example, a mobile device can perform a combination of at least one of (1) temperature check, (2) locating a digital vaccination certification from a public database (e.g., state databases), (3) collecting illness risk data using OCR and sharing them with an enterprise server, (4) storing digital vaccination certificates or digital version of vaccination cards, (5) communicating a barcode (e.g., QR code) with an enterprise server, (6) providing answers to a symptoms questionnaire and sharing them with an enterprise server, and/or (7) collecting test results of a particular illness and sharing them with an enterprise server.

Second, implementations in the present disclosure can allow an enterprise server (e.g., through a web application) to control illness risk information of employees/visitors such that the server can measure vital information of employees/visitors (as a category of illness risk information), integrate the illness risk information with HR platform, manage the illness risk information and/or wellness information, and control notifications relating to the illness risk information (e.g., send a quarantine notification to a mobile device of a user).

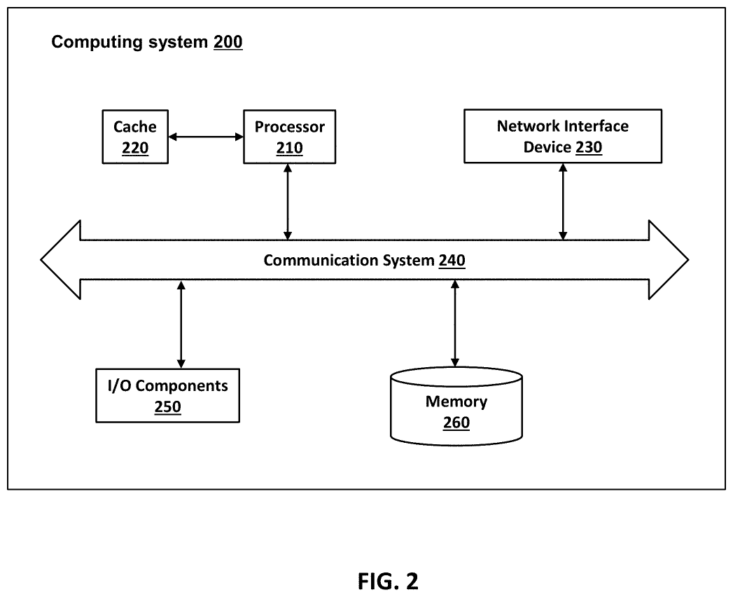

FIG. 1 is a block diagram illustrating an example of a system environment for an illness information control system 100 according to some implementations. In the illness information control system 100, a temperature scanner 130 is connected with a mobile device 150 via a network. For example, the temperature scanner 130 and the mobile device 150 may communicate with each other via Bluetooth or via Wi-Fi connection 172, 174 through a local access point 170). In some implementations, a mobile device 131 may be equipped with a temperature scanner. The mobile device 131, 150 may have configuration similar to that of a computing system 200 (see FIG. 2), for example, a smartphone or a tablet computer. In some implementations, the illness information control system 100 may be configured to monitor a body temperature of a person using a temperature sensor of the temperature scanner 130, share the temperature as illness risk information 192, 194 with a server 110 and a mobile device 131, 150, and display the temperature in the form of image or text on the mobile device 150 or a server 110. As shown in FIG. 1, the illness risk information 192, 194 may include information relating to (1) a temperature of a person (e.g., visitor, employee, etc.) as obtained from a mobile device 131, (2) a response of the person to symptom questionnaires as inputted from a mobile device 131, 150, (3) vaccination administration data of the person as inputted from a mobile device 131, 150, or (4) test results of the person as inputted from a mobile device 131, 150.

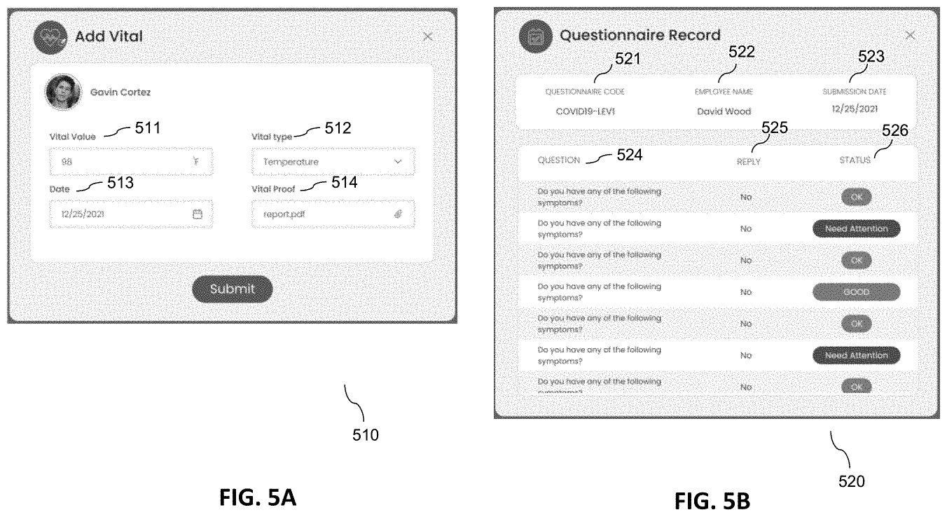

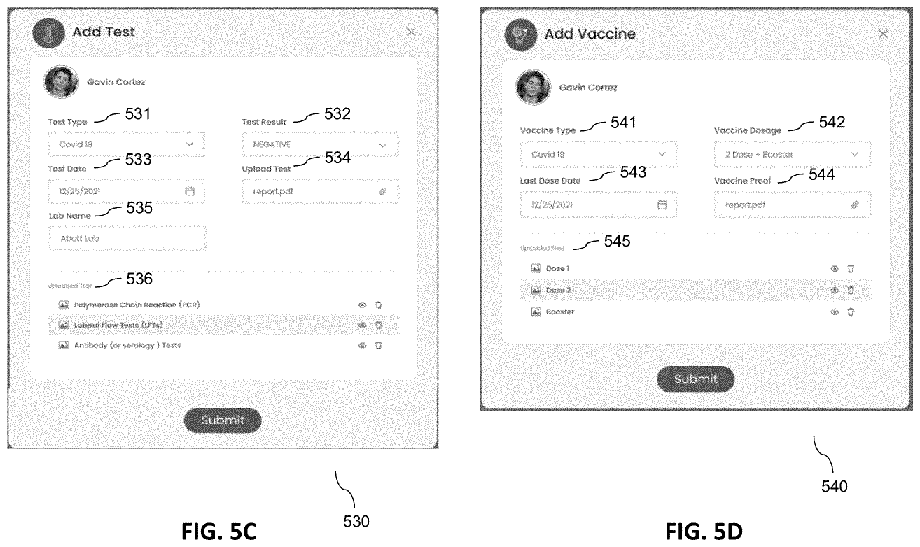

The illness information control system 100 may be configured to determine, based on illness risk information, whether entry of a person is permitted or the person has an illness risk or the person is to be in quarantine. For example, regarding a particular illness with respect to a particular person, the illness information control system 100 may be configured to determine whether entry of the person is permitted or the person has an illness risk or the person is to be in quarantine, based on illness risk information. The illness risk information may include vital information of the person (e.g. results of temperature measurement; see FIG. 5A), information relating to responses of the person to an illness questionnaire (e.g., questions relating to symptoms of the particular illness; see FIG. 5B), information relating to results of tests of the particular illness (see FIG. 5C), and/or information relating to administration of vaccine of the particular illness (see FIG. 5D). In some implementations, the illness information control system 100 may be connected to a server 110. The server 110 may have configuration similar to that of a computing system 200 (see FIG. 2). For example, the server 110 may be a remote cloud server. In some implementations, the illness information control system 100 may be connected to an external public database 120. For example, the public database 120 is a state or local vaccination database.

FIG. 2 is a block diagram illustrating an example of a computing system according to some implementations.

Referring to FIG. 2, the illustrated example computing system 200 includes one or more processors 210 in communication, via a communication system 240 (e.g., bus), with memory 260, at least one network interface controller 230 with network interface port for connection to a network (not shown), and other components, e.g., an input/output (“I/O”) components interface 450 connecting to a display (not illustrated) and an input device (not illustrated). Generally, the processor(s) 210 will execute instructions (or computer programs) received from memory. The processor(s) 210 illustrated incorporate, or are directly connected to, cache memory 220. In some instances, instructions are read from memory 260 into the cache memory 220 and executed by the processor(s) 210 from the cache memory 220.

In more detail, the processor(s) 210 may be any logic circuitry that processes instructions, e.g., instructions fetched from the memory 260 or cache 220. In some implementations, the processor(s) 210 are microprocessor units or special purpose processors. The computing device 200 may be based on any processor, or set of processors, capable of operating as described herein. The processor(s) 210 may be single core or multi-core processor(s). The processor(s) 210 may be multiple distinct processors.

The memory 260 may be any device suitable for storing computer readable data. The memory 260 may be a device with fixed storage or a device for reading removable storage media. Examples include all forms of non-volatile memory, media and memory devices, semiconductor memory devices (e.g., EPROM, EEPROM, SDRAM, and flash memory devices), magnetic disks, magneto optical disks, and optical discs (e.g., CD ROM, DVD-ROM, or Blu-Ray® discs). A computing system 200 may have any number of memory devices as the memory 260.

The cache memory 220 is generally a form of computer memory placed in close proximity to the processor(s) 210 for fast read times. In some implementations, the cache memory 220 is part of, or on the same chip as, the processor(s) 210. In some implementations, there are multiple levels of cache 220, e.g., L2 and L3 cache layers.

The network interface controller 230 manages data exchanges via the network interface (sometimes referred to as network interface ports). The network interface controller 230 handles the physical and data link layers of the OSI model for network communication. In some implementations, some of the network interface controller's tasks are handled by one or more of the processor(s) 210. In some implementations, the network interface controller 230 is part of a processor 210. In some implementations, a computing system 200 has multiple network interfaces controlled by a single controller 230. In some implementations, a computing system 200 has multiple network interface controllers 230. In some implementations, each network interface is a connection point for a physical network link (e.g., a cat-5 Ethernet link). In some implementations, the network interface controller 230 supports wireless network connections and an interface port is a wireless (e.g., radio) receiver/transmitter (e.g., for any of the IEEE 802.11 protocols, near field communication “NFC”, Bluetooth, ANT, or any other wireless protocol). In some implementations, the network interface controller 230 implements one or more network protocols such as Ethernet. Generally, a computing device 200 exchanges data with other computing devices via physical or wireless links through a network interface. The network interface may link directly to another device or to another device via an intermediary device, e.g., a network device such as a hub, a bridge, a switch, or a router, connecting the computing device 200 to a data network such as the Internet.

The computing system 200 may include, or provide interfaces for, one or more input or output (“I/O”) devices 250. Input devices include, without limitation, keyboards, microphones, touch screens, foot pedals, sensors, MIDI devices, and pointing devices such as a mouse or trackball. Output devices include, without limitation, video displays, speakers, refreshable Braille terminal, lights, MIDI devices, and 2-D or 3-D printers.

Other components may include an I/O interface, external serial device ports, and any additional co-processors. For example, a computing system 200 may include an interface (e.g., a universal serial bus (USB) interface) for connecting input devices, output devices, or additional memory devices (e.g., portable flash drive or external media drive). In some implementations, a computing device 200 includes an additional device such as a co-processor, e.g., a math co-processor can assist the processor 210 with high precision or complex calculations.

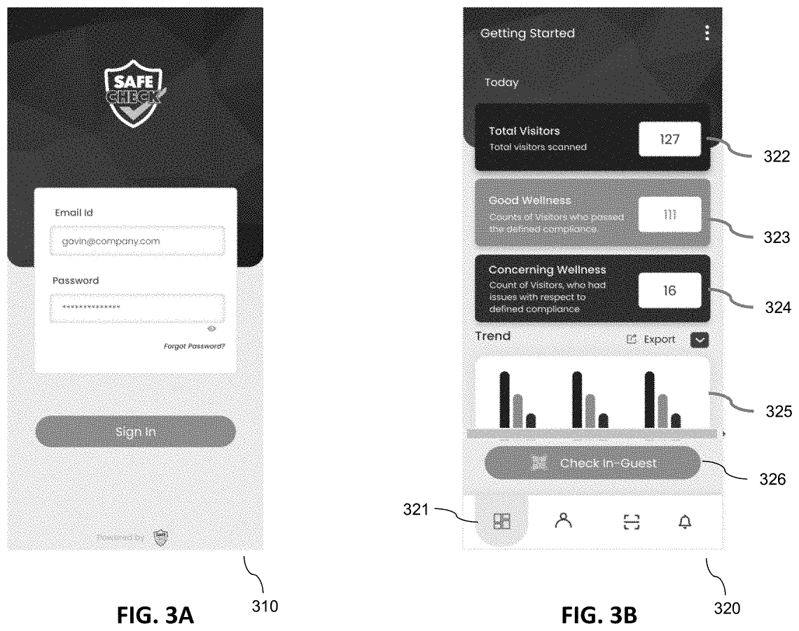

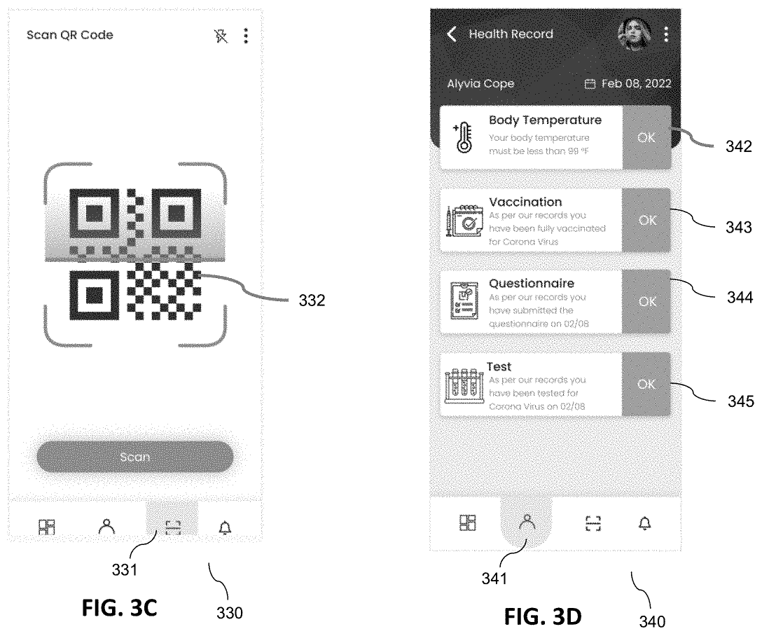

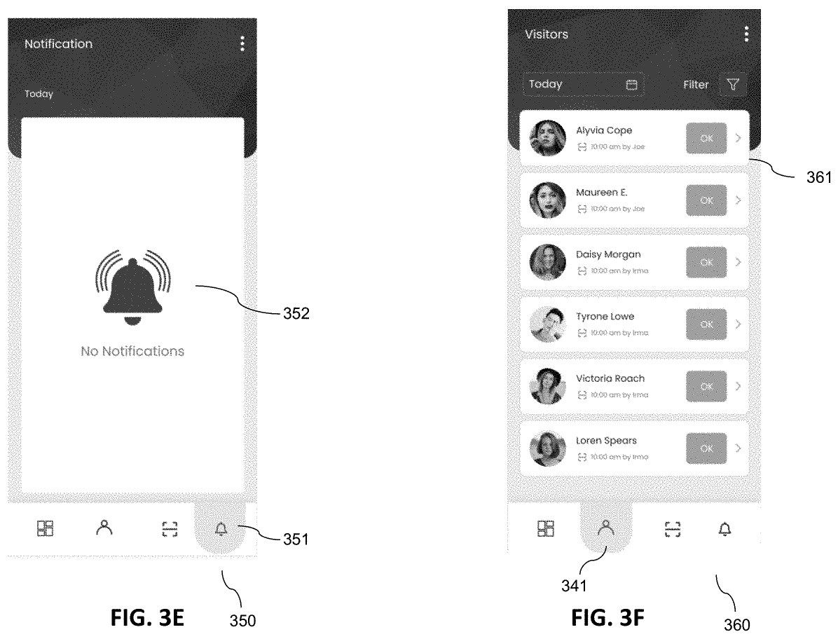

FIG. 3A to FIG. 3F are example user interfaces for displaying illness information on a mobile device according to some implementations. In some implementations, referring to FIG. 3A and FIG. 3B, the mobile device may display a log-in interface 310 to allow a user (e.g., an administrator of an illness information control system) to login an app (e.g., visitor admin app) and display a dashboard interface 320 to show a total number of visitors 322, a number of visitors with good wellness 323, a number of visitors with illness risk 324, and/or bar charts 325 showing trends of a breakdown of visitors (e.g., total number of visitors, number of good wellness, number of illness risk). In some implementations, the mobile device may display the dashboard interface 320 upon selection or clicking of a dashboard tab 321. Upon selection or clicking of a check in guest button 326, the mobile device may display a barcode (or QR code) scan interface 330 shown in FIG. 3C.

When a visitor user logs into an app (e.g., wellness app or visitor app) on his or her mobile device (not shown), the visitor user can generate a QR code (e.g., QR code 332 in FIG. 3C) on his or her mobile device. Referring to FIG. 3C, in the scan interface 330, the mobile device of the administrator may scan the QR code 332 displayed on the mobile device of the visitor user. In some implementations, the mobile device may display the scan interface 330 upon selection or clicking of a scan tab 331.

Referring to FIG. 3D, the administrator mobile device may identify the visitor user using the QR code and display a health record interface 340 to show illness risk information of that visitor user. The illness risk information may include one or more categories including (1) vital information 342 (e.g., temperature), (2) vaccination information 343, (3) symptom questionnaire information 344 and/or (4) test result information 345, and whether each category of the illness risk information passes a predetermined criterion (“OK”) or not. In some implementations, the mobile device may display the health record interface 340 upon selection or clicking of a user tab 341.

Referring to FIG. 3E, upon selection or clicking of a notification tab 351, the mobile device may display a notification interface 350 to show any notification on the current user (e.g., notification relating to an illness risk or quarantine) or indicate no notifications 352.

Referring to FIG. 3F, upon selection or clicking of the user tab 341 without selecting the check in guest button 326 (without performing a guest check in process or a QR code scanning process), the mobile device may display a visitor list interface 360 showing a list of visitors 361 and whether each visitor passes predetermined illness risk criteria (“OK”) or not.

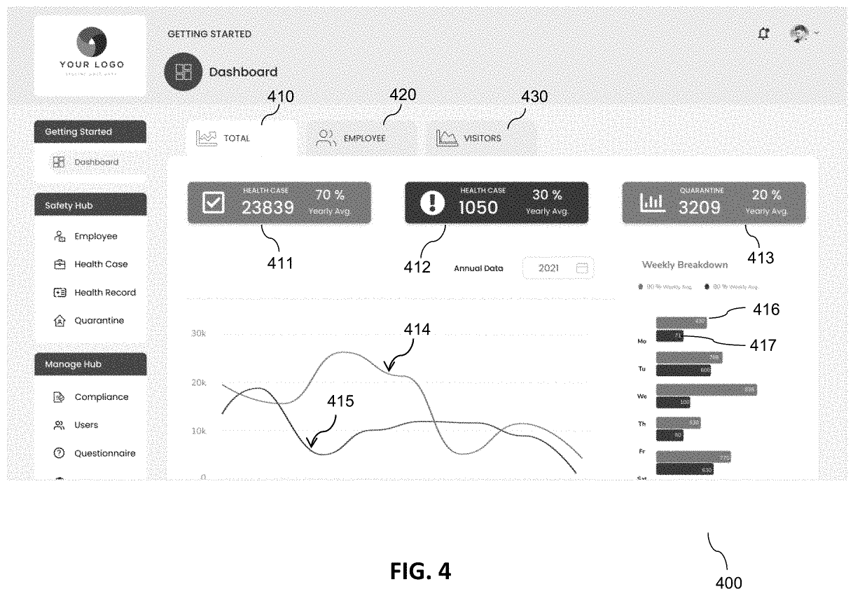

FIG. 4 is an example user interface for displaying illness information for an enterprise environment according to some implementations.

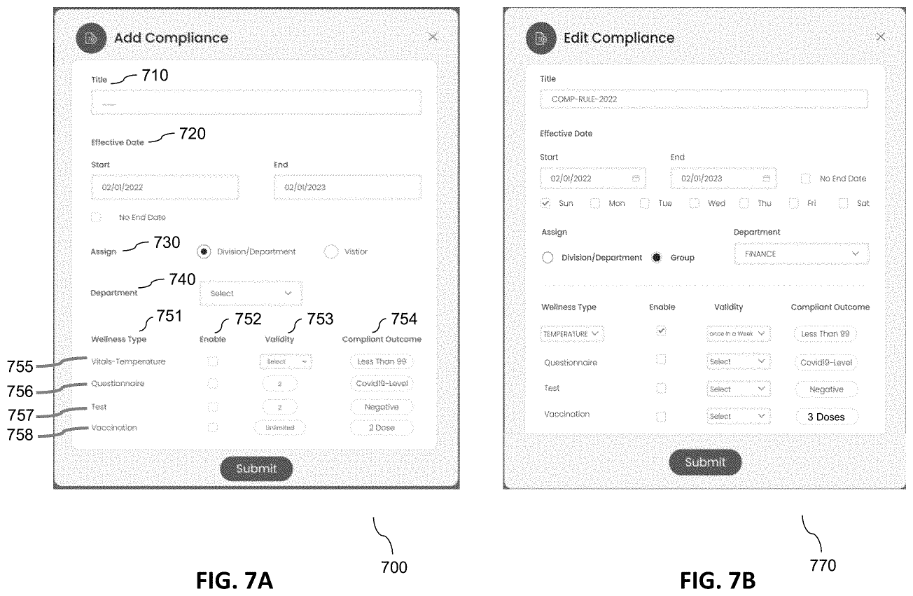

In some implementations, an illness information control system (e.g., system 100 in FIG. 1) may allow a server device (e.g., server device 110) or a mobile device (e.g., mobile device 131, 150 in FIG. 1) to provide a dashboard or console interface 400 to display data related with the number of good health cases (with no illness risks) 411, the number of bad health cases (with illness risks) 412, and the number of quarantine cases 413 among the number of people in a total tab 410 during a particular period (e.g., year 2021). In some implementations, the system may determines whether a person has a good health case 411, a bad health case 412, or a quarantine case 413, based on one or more of different categories of illness risk information of that person (e.g., those shown in FIG. 5A to FIG. 5D). In some implementations, the system may determines whether a person has a good health case 411, a bad health case 412, or a quarantine case 413, based on predetermined criteria (e.g., compliance shown in FIG. 7A and FIG. 7B) on one or more of different categories of illness risk information of that person.

The interface 400 may include (1) a line chart indicating the number of good health cases 414 and the number of bad health cases 415 over time, and (2) a bar chart indicating a weekly breakdown of the number of good health cases (e.g., bar 416 on Monday) and the number of bad health cases (e.g., bar 417 on Monday). In this manner, the illness information control system can share a plurality of sources of illness risk data (e.g., temperature, questionnaire response, vaccination, test results) of a person in real time, and combine and display them in real time as shown in the interface 400 in FIG. 4. Upon selection of an employee tab 420, the system may provide a similar interface to display data among the number of employees during the particular period. Upon selection of a visitors tab 430, the system may provide a similar interface to display data among the number of visitors during the particular period.

FIG. 5A to FIG. 5D are example user interfaces for receiving or inputting different categories of illness information from users according to some implementations.

FIG. 5A is an example user interface 510 for receiving vital information of a particular user and storing the vital information in a storage (e.g., database). For example, an illness information control system (e.g., system 100 in FIG. 1) may allow a server device (e.g., server device 110) or a mobile device (e.g., mobile device 131, 150 in FIG. 1) to receive temperature measurement data as vital information from a temperature scanner (e.g., scanner 130 or mobile device 131 in FIG. 1). The temperature measurement data may include a vital value (e.g., temperature value) 511, a vital type (e.g., temperature type) 512, measurement date 513, proof document (e.g., document including temperature measurement data) 514.

FIG. 5B is an example user interface 520 for receiving questionnaire response information of a particular user and storing the response information in a storage (e.g., database). For example, an illness information control system (e.g., system 100 in FIG. 1) may allow a server device (e.g., server device 110) or a mobile device (e.g., mobile device 131, 150 in FIG. 1) to receive questionnaire response data including answers or responses 525 of a particular user 522 to one or more questions 524 in a predetermined type of questionnaire 521, from a mobile device (e.g., mobile device 131 or 150 in FIG. 1) on a particular date 523. In some implementations, the predetermined type of questionnaire 521 may relate to symptoms of a particular illness (e.g., COVID-19). The system may compare the responses of the particular users with predetermined model responses, and determine as status 526 of each response whether each response indicates a good health (with no illness risks; “GOOD”), a normal health (with a low illness risk; “OK”), or a bad health (with a high illness risk; “Need Attention”).

FIG. 5C is an example user interface 530 for receiving information on test results of a particular user with respect to a particular illness (e.g., COVID-19) and storing the test result information in a storage (e.g., database). For example, an illness information control system (e.g., system 100 in FIG. 1) may allow a server device (e.g., server device 110) or a mobile device (e.g., mobile device 131, 150 in FIG. 1) to receive test result data from a mobile device of the particular user. For example, the particular user may upload test result data (e.g., digital document including test result data) to the system from the mobile device of the particular user (e.g., through a wellness app). The test result data may include a test type 531 (e.g., test relating to a particular illness), a test result (e.g., positive or negative) 532, a test date 533, an uploaded document 534 (e.g., document in the format of PDF, Word, JPEG, JPG, GIF, PNG, etc.), a test location or laboratory 535, and/or test results 536 that have been previously uploaded.

FIG. 5D is an example user interface 540 for receiving information on vaccine administrations of a particular user with respect to a particular illness (e.g., COVID-19) and storing the vaccine administration information in a storage (e.g., database). For example, an illness information control system (e.g., system 100 in FIG. 1) may allow a server device (e.g., server device 110) or a mobile device (e.g., mobile device 131, 150 in FIG. 1) to receive vaccine administration data from a mobile device of the particular user. For example, the particular user may upload vaccine administration data (e.g., digital document including vaccine administration data) to the system from the mobile device of the particular user (e.g., through a wellness app). The vaccine administration data may include a vaccine type 541 (e.g., vaccine relating to a particular illness), a vaccine dosage (e.g., number of dosages administered or booster administration) 542, last administration date 543, an uploaded vaccine proof 544 (e.g., vaccination card in the format of PDF, Word, JPEG, JPG, GIF, PNG, etc.), and/or vaccine proofs 545 that have been previously uploaded.

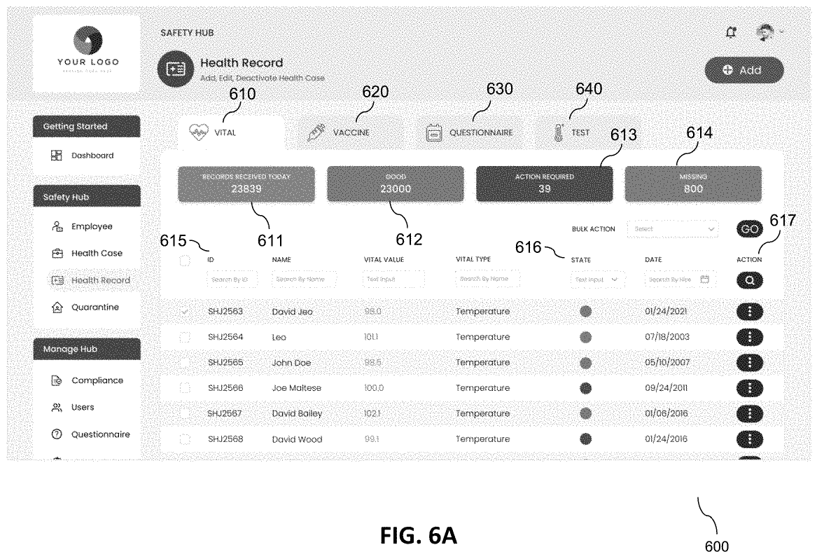

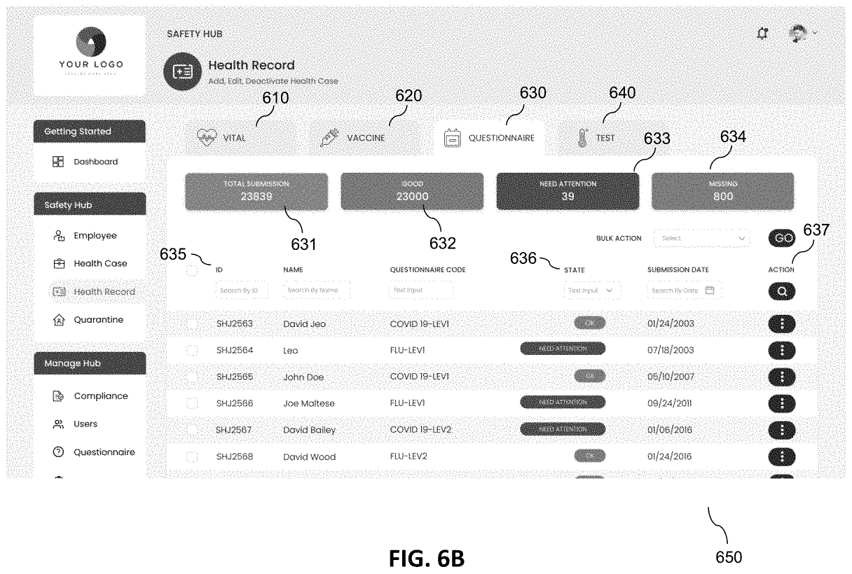

FIG. 6A and FIG. 6B are example user interfaces for displaying different categories of illness information according to some implementations.

In some implementations, an illness information control system (e.g., system 100 in FIG. 1) may allow a server device (e.g., server device 110) or a mobile device (e.g., mobile device 131, 150 in FIG. 1) to provide a health record interface 600 to display data related with the total number of vital records received today 611, the number of good vital records (e.g., temperature value indicating no illness risks) 612, the number of bad vital records (e.g., temperature value indicating illness risks; “Action Required”) 613, and the number of missing vital records 614 in a vital tab 610. In some implementations, the system may list vital records of employees including employee ID 615 of a particular person, a state 616 of a particular vital record of the particular person, and action 617 that can be performed on the particular vital record. For example, the state 616 may be either “Good”, “Action Required”, or “Missing”. The action 617 may include sending to the particular person an illness risk notification (if the state 616 is “Action Required”) or a missing notification (if the state 616 is “Missing”).

Referring to FIG. 6B, the system may allow a server device (e.g., server device 110) or a mobile device (e.g., mobile device 131, 150 in FIG. 1) to provide a health record interface 650 to display data related with the total number of questionnaire records received today 631, the number of good questionnaire records (e.g., responses thereof indicating no illness risks) 632, the number of bad questionnaire records (e.g., responses thereof indicating illness risks; “Need Attention”) 633, and the number of missing questionnaire records 634 in the questionnaire tab 630. In some implementations, the system may list questionnaire records of employees including employee ID 635 of a particular person, a state 636 of a particular questionnaire record of the particular person, and action 637 that can be performed on the particular questionnaire record. For example, the state 636 may be either “Good”, “Need Attention”, or “Missing”. The action 637 may include sending to the particular person an illness risk notification (if the state 636 is “Need Attention”) or a missing notification (if the state 636 is “Missing”).

Upon selection of another tab (e.g., vaccine tab 620, test tab 640), the system may provide a similar interface to display data relating to corresponding records (e.g., vaccine records, test result records). For example, upon selection of the vaccine tab 620, the system may list the vaccine records of employees showing that a state of a particular vaccine record is either “Fully Vaccinated”, “Partially Vaccinated”, or “Not Vaccinated.” Similarly, upon selection of the test tab 640, the system may list the test result records of employees showing that a state of a particular test result record is either “Test Negative”, “Test Positive”, or “Missing Reports.”

FIG. 7A and FIG. 7B are example user interfaces for adding and editing compliance criteria on different categories of illness information according to some implementations.)

In some implementations, an illness information control system (e.g., system 100 in FIG. 1) may allow a server device (e.g., server device 110) or a mobile device (e.g., mobile device 131, 150 in FIG. 1) to provide a compliance add interface 700 to set predetermined criteria as a compliance on one or more of different categories of illness risk information received from users (e.g., vital data, questionnaire (response) data, test result data, vaccine administration data as shown in FIG. 5A to FIG. 5D). The system may determine whether a person has a good health case, a bad health case, or a quarantine case (see FIG. 4) based on the predetermined criteria.

Referring to FIG. 7A, the system may add a new compliance (e.g., criteria on one or more categories of illness risk information) including title 710 of the compliance, effective dates 720 of the compliance including start date and end date, assignment 730 indicating to which division/department or visitor the compliance is assigned (in other words, the compliance criteria is to be applied to the selected one of division/department or visitor), department 740 to which the compliance is assigned, a criterion 755 for vital information, a criterion 756 for questionnaire response information, a criterion 757 for test result information, and/or a criterion 758 for vaccine administration information. Each of the criteria 755, 756, 757, 758 may include wellness type 751 (e.g., vital information, questionnaire response information, test result information, or vaccine administration information), enabled checkbox 751 indicating the corresponding wellness type is included in the compliance criteria, validity 753 indicating frequency or counts to be valid (e.g., temperature measurement needs to be performed once in a week, questionnaire needs to be submitted at least twice, test result needs to be submitted at least twice, vaccination records may be submitted an “unlimited” number of times), and compliance condition 754 indicating a condition that should be met to be a “good” health case. For example, the system may determine that a particular person meets the compliance as set in FIG. 7A (or the person is a “good” or “OK” health case), if all of the criteria 755, 756, 757, 758 are satisfied such that (1) a temperature of the particular person is less than 99, (2) responses of the particular person to a symptom questionnaire qualify for a COVID-19 level (e.g., the person has no symptoms of COVID-19), (3) the most recent test result of the particular person is negative, and (4) at least two doses of vaccine have been administered to the person. On the other hand, the system may determine that a particular person does not meet the compliance as set in FIG. 7A (or the person is a “concerning” health case), if at least one of the criteria 755, 756, 757, 758 is not satisfied such that (1) a temperature of the particular person is greater than or equal to 99, or (2) responses of the particular person to a symptom questionnaire do not qualify for a COVID-19 level (e.g., the person has at least one symptom of COVID-19), or (3) the most recent test result of the particular person is positive, or (4) only one dose of vaccine or no vaccine has been administered to the person. The system may determine that a particular person is a “bad” health case or an action is required for the particular person, if none of the temperature criterion 755 and the test result criterion 757 are satisfied such that (1) a temperature of the particular person is greater than or equal to 99, and (3) the most recent test result of the particular person is positive. The system may determine that a particular person should be in quarantine, if the test result criterion 757 is not satisfied such that (3) the most recent test result of the particular person is positive.

Referring to FIG. 7B, an illness information control system (e.g., system 100 in FIG. 1) may allow a server device (e.g., server device 110) or a mobile device (e.g., mobile device 131, 150 in FIG. 1) to provide a compliance edit interface 770 to edit or modify predetermined criteria as a compliance on one or more of different categories of illness risk information. The compliance edit interface 770 may have fields corresponding to those of the compliance add interface 700 so that the server device or the mobile device can change values of the compliance fields to set different criteria as an edited compliance on the one or more of different categories of illness risk information. For example, the compliance condition 754 for the vaccine criterion 758 can be changed such that the system may determine that a particular person meets the compliance (or the person is a “good” or “OK” health case), if a temperature of the particular person is less than 99, responses of the particular person to a symptom questionnaire qualify for a COVID-19 level, the most recent test result of the particular person is negative, and at least three doses of vaccine have been administered to the person.

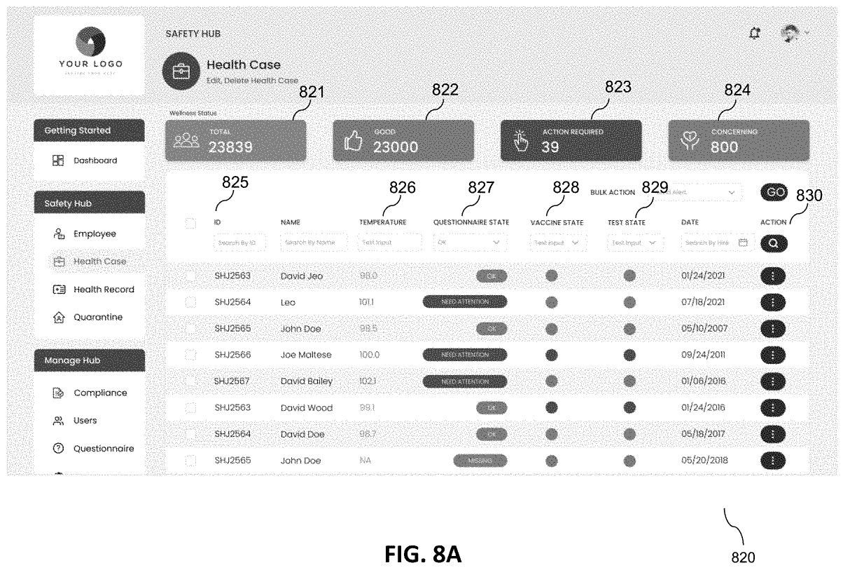

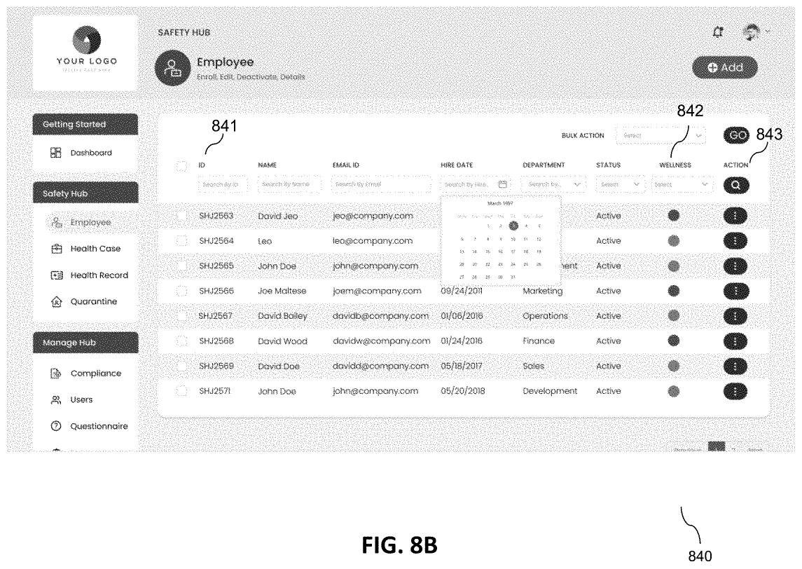

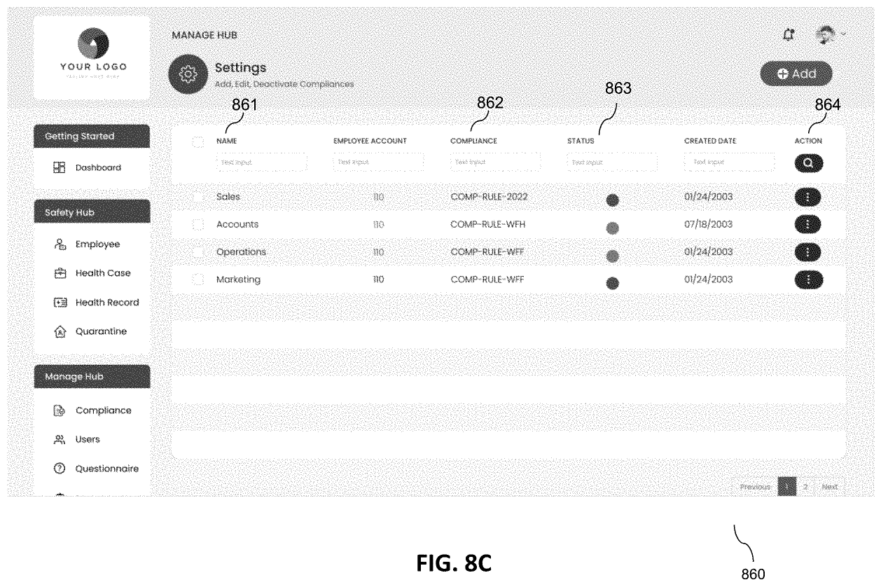

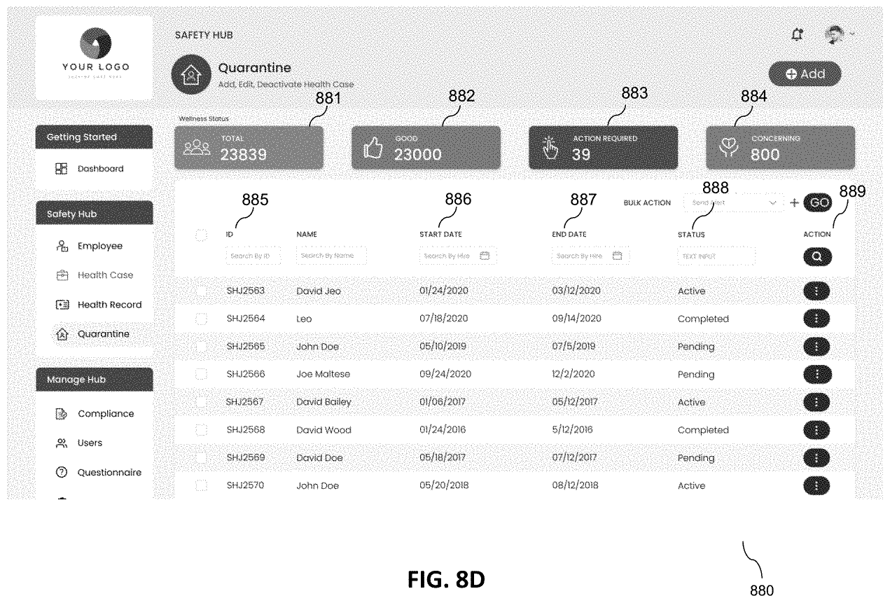

FIG. 8A to FIG. 8D are example user interfaces for displaying illness information based on one or more compliances (e.g., compliances as shown in FIG. 7A and FIG. 7B) according to some implementations.

Referring to FIG. 8A, an illness information control system (e.g., system 100 in FIG. 1) may allow a server device (e.g., server device 110) or a mobile device (e.g., mobile device 131, 150 in FIG. 1) to provide a health case interface 820 to display data related with the total number of health cases 821, the number of “good” health cases 822, the number of “bad” health cases (or “Action Required” cases) 823, and the number of “concerning” health cases 824, which may be determined based on predetermined criteria (e.g., criteria 755, 756, 757, 758 as shown in FIG. 7A). In some implementations, the system may list health cases of employees including employee ID 825 of a particular person, a temperature 826 of a particular health case of the particular person, a questionnaire state 827 of the particular health case of the particular person (e.g., “OK”, “Need Attention” or “Missing”), a vaccine state 828 of the particular health case of the particular person (e.g., “Fully Vaccinated”, “Partially Vaccinated”, or “Not Vaccinated”), a test result state 829 of the particular health case of the particular person (e.g., “Test Negative”, “Test Positive”, or “Missing Reports”), and an action 830 that can be performed on the particular case depending on the state of that case (e.g., sending to the particular person an illness risk notification or a record missing notification or a quarantine notification).

Referring to FIG. 8B, an illness information control system (e.g., system 100 in FIG. 1) may allow a server device (e.g., server device 110) or a mobile device (e.g., mobile device 131, 150 in FIG. 1) to provide an employee wellness interface 840 to list health information of employees including employee ID 841 of a particular person, a wellness state 842 of the particular person (e.g., “good”, “bad (or action required)” or “concerning” health cases, determined based on the criteria 755, 756, 757, 758 as shown in FIG. 7A), and an action 843 that can be performed on the particular case depending on the wellness state of that case (e.g., sending to the particular person an illness risk notification or a quarantine notification).

Referring to FIG. 8C, an illness information control system (e.g., system 100 in FIG. 1) may allow a server device (e.g., server device 110) or a mobile device (e.g., mobile device 131, 150 in FIG. 1) to provide a department wellness interface 860 to list health information of employees of respective departments, including department name 861 of a particular department, a compliance or criteria 862 used for the particular department, a wellness state 863 of the employees of the particular department (e.g., “good”, “bad (or action required)” or “concerning” health cases, determined based on the compliance or criteria 862 as applied to the employees of the particular department), and an action 864 that can be performed on the particular department depending on the wellness state of that department (e.g., sending to the particular department (to the department head) an illness risk notification or a quarantine notification). In some implementations, wellness state 863 may be determined by calculating an average wellness state of wellness states of all the employees of the particular department based on the compliance or criteria 862.

Referring to FIG. 8D, an illness information control system (e.g., system 100 in FIG. 1) may allow a server device (e.g., server device 110) or a mobile device (e.g., mobile device 131, 150 in FIG. 1) to provide a quarantine interface 880 to display data related with the total number of health cases 881, the number of “good” health cases 882, the number of “bad” health cases (or “Action Required” cases) 883, and the number of “concerning” health cases 884 in a manner similar to those shown in FIG. 8A. In some implementations, the system may list quarantine cases of employees including employee ID 885 of a particular person, a quarantine start date 886 of a particular quarantine case of the particular person, a quarantine end date 887 of the particular quarantine case of the particular person, a quarantine state 888 of the particular quarantine case of the particular person (e.g., “active” indicating that the particular person is currently in quarantine, “completed” indicating that the particular person has completed a quarantine, or “pending” indicating that the particular person is determined to be in quarantine but has not been in quarantine), and an action 830 that can be performed on the particular case depending on the state of that case (e.g., sending to the particular person a quarantine notification including start date and end date of a quarantine).

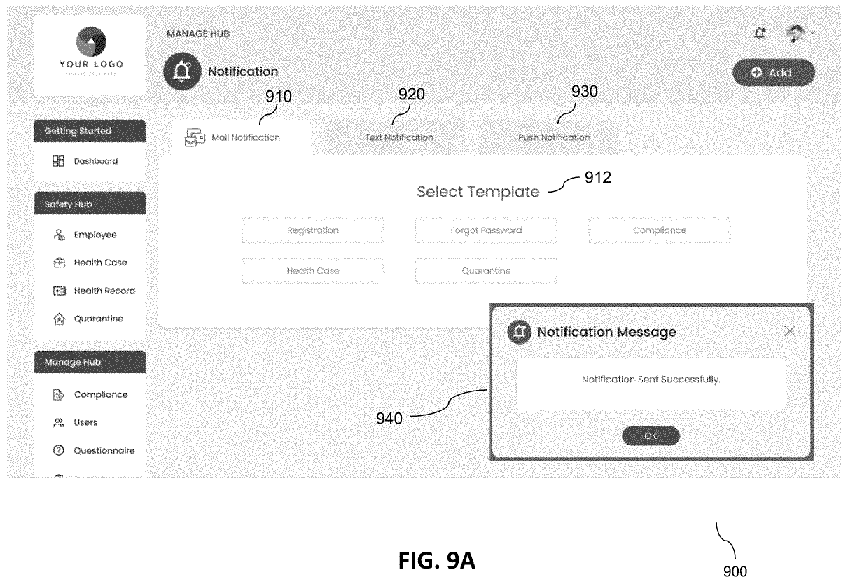

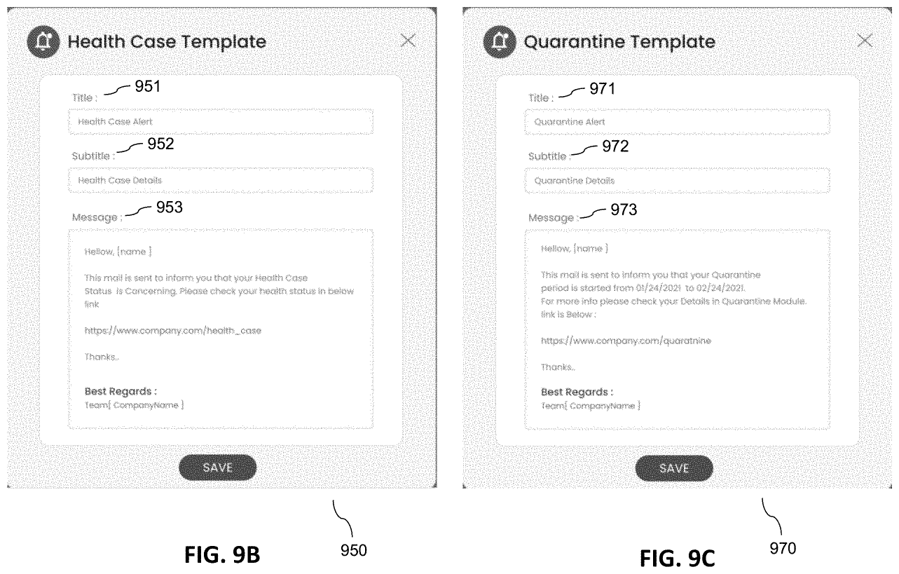

FIG. 9A to FIG. 9C are example user interfaces for controlling notifications relating to illness information according to some implementations.

Referring FIG. 9A, an illness information control system (e.g., system 100 in FIG. 1) may allow a server device (e.g., server device 110) or a mobile device (e.g., mobile device 131, 150 in FIG. 1) to provide a notification interface 900 to display notification templates 912 for selection in a mail notification tab 410. In some implementations, the selectable notification templates 912 may include templates for notifying (1) registration (e.g., requiring a particular user to register the user's profile as soon as possible), (2) forgot password (e.g., sending a link to change the password of a particular user), (3) compliance (e.g., informing that a new compliance for employee has been created), (4) health case, and/or (5) quarantine. Upon selection of another tab (e.g., a text notification tab 920, a push notification tab 930), the system may provide a similar notification interface to display notification templates for selection in the corresponding notification method (e.g., text (SMS) notification, push notification (clickable pop-up messages)).

Referring to FIG. 9B, the system may allow the server device or the mobile device to display a health case template 950 to send a health case notification to a particular user. For example, the health case template 950 may include a title 951, a subtitle 952, and a message 953 of the health case notification. The message 953 may indicate a health state of the particular user and a link to display detailed information of the health state. Similarly, referring to FIG. 9C, the system may allow the server device or the mobile device to display a quarantine template 970 to send a quarantine notification to a particular user. For example, the quarantine template 970 may include a title 971, a subtitle 972, and a message 973 of the quarantine notification. The message 973 may indicate a period of a particular quarantine of the particular user and a link to display detailed information of the particular quarantine.

Referring back to FIG. 9A, after selecting a template from notification templates 912 and filling in the selected template (e.g., filling in the title, subtitle and message fields of the selected template as shown in FIG. 9B and FIG. 9C), the system may allow the server device or the mobile device to send the filled-in notification to a particular user. Upon successfully sending the notification, the system may display a successful notification message 940 in a sender's device (see FIG. 9A), while displaying the received notification in a receiver's device (e.g., displaying the notification upon selecting the notification tab 351 of the receiver's mobile device as shown in FIG. 3E).

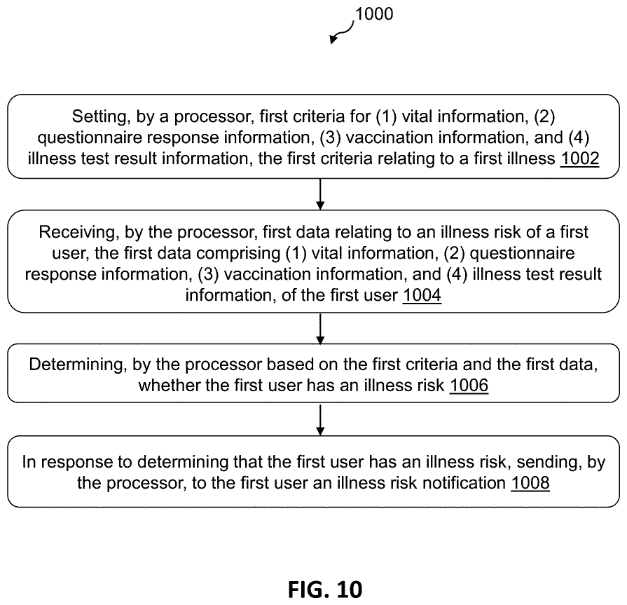

FIG. 10 is a flowchart illustrating an example methodology for controlling notifications relating to illness information according to some implementations.

In this example methodology, the process 1000 begins at step 1002 by setting, by a processor (e.g., processor 210 in FIG. 2), first criteria (e.g., criteria 755, 756, 757, 758 in FIG. 7A) for (1) vital information (e.g., temperature information added in user interface 510 in FIG. 5A), (2) questionnaire response information (e.g., questionnaire response information shown in user interface 520 in FIG. 5B), (3) vaccination information (e.g., vaccination information added in user interface 540 in FIG. 5D), and (4) illness test result information (e.g., test result information added in user interface 530 in FIG. 5C). The first criteria (e.g., criteria 755, 756, 757, 758 in FIG. 7A) may relate to a first illness (e.g., COVID-19).

In some implementations, in setting the first criteria, the processor may be configured to set a criterion for each of (1) vital information (e.g., temperature criterion 755 in FIG. 7A), (2) questionnaire response information (e.g., questionnaire criterion 756 in FIG. 7A), (3) vaccination information (e.g., vaccination criterion 758 in FIG. 7A), and (4) illness test result information (e.g., test result criterion 757 in FIG. 7A). In setting the first criteria, the processor may be configured to set a group of users to which the first criteria are to be applied (e.g., assignment 730 to division/department 740 in FIG. 7A), wherein the group of users includes the first user.

In some implementations, the processor may be configured to set second criteria for (1) vital information, (2) questionnaire response information, (3) vaccination information, and (4) illness test result information, the second criteria relating to a second illness different from the first illness. For example, the system may set different compliances or criteria (e.g., different compliances 862 including COMP-RULE-2022, COMP-RULE-WFH, COMP-RULE-WFF as shown in FIG. 8C).

At step 1004, in some implementations, the processor may receive first data relating to an illness risk of a first user, the first data comprising (1) vital information (e.g., temperature information added in user interface 510 in FIG. 5A), (2) questionnaire response information (e.g., questionnaire response information shown in user interface 520 in FIG. 5B), (3) vaccination information (e.g., vaccination information added in user interface 540 in FIG. 5D), and (4) illness test result information (e.g., test result information added in user interface 530 in FIG. 5C), of the first user.

In some implementations, the vital information of the first user may include a measured body temperature of the first user (e.g., vital type 512 is temperature in FIG. 5A). In some implementations, the questionnaire response information of the first user may include one or more responses (e.g., replies or responses 525 in FIG. 5B) to one or more questions (e.g., questions 524 in FIG. 5B) relating to symptoms of the first illness (e.g., symptoms of COVID-19). In some implementations, the vaccination information of the first user may include information relating to administration of one or more doses of vaccine of the first illness to the first user (e.g., vaccine dosage 542 in FIG. 5D). In some implementations, the illness test result information of the first user may include information relating to administration of one or more tests of the first illness to the first user (e.g., test type 531, test result 532, test date 533, test laboratory 535 in FIG. 5C).

At step 1006, in some implementations, the processor may determine, based on the first criteria and the first data, whether the first user has an illness risk. In determining whether the first user has an illness risk, the processor may be configured to compare each of the information (1) to (4) of the first data with the corresponding criterion, and determine, based on a result of the comparison, whether the first user has an illness risk. For example, an illness information control system (e.g., system 100 in FIG. 1) may determine that a particular person meets the compliance as set in FIG. 7A (the person is a “good” or “OK” health case and has no illness risk), if all of the criteria 755, 756, 757, 758 are satisfied such that (1) a temperature of the particular person is less than 99, (2) responses of the particular person to a symptom questionnaire qualify for a COVID-19 level (e.g., the person has no symptoms of COVID-19), (3) the most recent test result of the particular person is negative, and (4) the number of doses of vaccine administered to the person is greater than or equal to 2. On the other hand, the system may determine that a particular person does not meet the compliance as set in FIG. 7A (the person is a “concerning” health case and has some illness risk), if at least one of the criteria 755, 756, 757, 758 is not satisfied such that (1) a temperature of the particular person is greater than or equal to 99, or (2) responses of the particular person to a symptom questionnaire do not qualify for a COVID-19 level (e.g., the person has at least one symptom of COVID-19), or (3) the most recent test result of the particular person is positive, or (4) the number of doses of vaccine administered to the person is less than or equal to 1. The system may determine that a particular person is a “bad” health case and has a significant illness risk or an action is required for the particular person, if none of the temperature criterion 755 and the test result criterion 757 are satisfied such that (1) a temperature of the particular person is greater than or equal to 99, and (3) the most recent test result of the particular person is positive.

At step 1008, in some implementations, in response to determining that the first user has an illness risk, the processor may send to the first user an illness risk notification. For example, referring to FIG. 8B, if the wellness state 842 of a particular person is a “concerning” health case, determined based on the criteria 755, 756, 757, 758 as shown in FIG. 7A, the system may be configured to send to the particular person an illness risk notification (see FIG. 9A and FIG. 9B).

In some implementations, the processor may be configured to determine, based on the first criteria and the first data, whether the first user is to be in quarantine. Referring to FIG. 7A, the system may determine that a particular person should be in quarantine, if the test result criterion 757 is not satisfied such that (3) the most recent test result of the particular person is positive. In response to determining that the first user is to be in quarantine, the processor may be configured to send to the first user an illness quarantine notification including time and duration of quarantine. For example, referring to FIG. 8B, if the wellness state 842 of a particular person is a “bad (or action required)” health case, determined based on the criteria 755, 756, 757, 758 as shown in FIG. 7A, the system may be configured to send to the particular person a quarantine notification (see FIG. 9A and FIG. 9C).

The previous description is provided to enable any person skilled in the art to practice the various aspects described herein. Various modifications to these aspects will be readily apparent to those skilled in the art, and the generic principles defined herein may be applied to other aspects. Thus, the claims are not intended to be limited to the aspects shown herein, but is to be accorded the full scope consistent with the language claims, wherein reference to an element in the singular is not intended to mean “one and only one” unless specifically so stated, but rather “one or more.” Unless specifically stated otherwise, the term “some” refers to one or more. All structural and functional equivalents to the elements of the various aspects described throughout the previous description that are known or later come to be known to those of ordinary skill in the art are expressly incorporated herein by reference and are intended to be encompassed by the claims. Moreover, nothing disclosed herein is intended to be dedicated to the public regardless of whether such disclosure is explicitly recited in the claims. No claim element is to be construed as a means plus function unless the element is expressly recited using the phrase “means for.”

It is understood that the specific order or hierarchy of blocks in the processes disclosed is an example of illustrative approaches. Based upon design preferences, it is understood that the specific order or hierarchy of blocks in the processes may be rearranged while remaining within the scope of the previous description. The accompanying method claims present elements of the various blocks in a sample order, and are not meant to be limited to the specific order or hierarchy presented.

The previous description of the disclosed implementations is provided to enable any person skilled in the art to make or use the disclosed subject matter. Various modifications to these implementations will be readily apparent to those skilled in the art, and the generic principles defined herein may be applied to other implementations without departing from the spirit or scope of the previous description. Thus, the previous description is not intended to be limited to the implementations shown herein but is to be accorded the widest scope consistent with the principles and novel features disclosed herein.

The various examples illustrated and described are provided merely as examples to illustrate various features of the claims. However, features shown and described with respect to any given example are not necessarily limited to the associated example and may be used or combined with other examples that are shown and described. Further, the claims are not intended to be limited by any one example.

The foregoing method descriptions and the process flow diagrams are provided merely as illustrative examples and are not intended to require or imply that the blocks of various examples must be performed in the order presented. As will be appreciated by one of skill in the art the order of blocks in the foregoing examples may be performed in any order. Words such as “thereafter,” “then,” “next,” etc. are not intended to limit the order of the blocks; these words are simply used to guide the reader through the description of the methods. Further, any reference to claim elements in the singular, for example, using the articles “a,” “an” or “the” is not to be construed as limiting the element to the singular.

The various illustrative logical blocks, modules, circuits, and algorithm blocks described in connection with the examples disclosed herein may be implemented as electronic hardware, computer software, or combinations of both. To clearly illustrate this interchangeability of hardware and software, various illustrative components, blocks, modules, circuits, and blocks have been described above generally in terms of their functionality. Whether such functionality is implemented as hardware or software depends upon the particular application and design constraints imposed on the overall system. Skilled artisans may implement the described functionality in varying ways for each particular application, but such implementation decisions should not be interpreted as causing a departure from the scope of the present disclosure.

The hardware used to implement the various illustrative logics, logical blocks, modules, and circuits described in connection with the examples disclosed herein may be implemented or performed with a general purpose processor, a DSP, an ASIC, an FPGA or other programmable logic device, discrete gate or transistor logic, discrete hardware components, or any combination thereof designed to perform the functions described herein. A general-purpose processor may be a microprocessor, but, in the alternative, the processor may be any conventional processor, controller, microcontroller, or state machine. A processor may also be implemented as a combination of computing devices, e.g., a combination of a DSP and a microprocessor, a plurality of microprocessors, one or more microprocessors in conjunction with a DSP core, or any other such configuration. Alternatively, some blocks or methods may be performed by circuitry that is specific to a given function.

In some examples, the functions described may be implemented in hardware, software, firmware, or any combination thereof. If implemented in software, the functions may be stored as one or more instructions or code on a non-transitory computer-readable storage medium or non-transitory processor-readable storage medium. The blocks of a method or algorithm disclosed herein may be embodied in a processor-executable software module which may reside on a non-transitory computer-readable or processor-readable storage medium. Non-transitory computer-readable or processor-readable storage media may be any storage media that may be accessed by a computer or a processor. By way of example but not limitation, such non-transitory computer-readable or processor-readable storage media may include RAM, ROM, EEPROM, FLASH memory, CD-ROM or other optical disk storage, magnetic disk storage or other magnetic storage devices, or any other medium that may be used to store desired program code in the form of instructions or data structures and that may be accessed by a computer. Disk and disc, as used herein, includes compact disc (CD), laser disc, optical disc, digital versatile disc (DVD), floppy disk, and Blu-ray disc where disks usually reproduce data magnetically, while discs reproduce data optically with lasers. Combinations of the above are also included within the scope of non-transitory computer-readable and processor-readable media. Additionally, the operations of a method or algorithm may reside as one or any combination or set of codes and/or instructions on a non-transitory processor-readable storage medium and/or computer-readable storage medium, which may be incorporated into a computer program product.

The preceding description of the disclosed examples is provided to enable any person skilled in the art to make or use the present disclosure. Various modifications to these examples will be readily apparent to those skilled in the art, and the generic principles defined herein may be applied to some examples without departing from the spirit or scope of the disclosure. Thus, the present disclosure is not intended to be limited to the examples shown herein but is to be accorded the widest scope consistent with the following claims and the principles and novel features disclosed herein.

What is claimed is:

1. A system for controlling notifications relating to illness information, comprising:
a processor and memory,
wherein the processor is configured to;
set first criteria for (1) vital information, (2) questionnaire response information, (3) vaccination information, and (4) illness test result information, the first criteria relating to a first illness;
receive first data relating to an illness risk of a first user, the first data comprising (1) vital information, (2) questionnaire response information, (3) vaccination information, and (4) illness test result information, of the first user;
determine, based on the first criteria and the first data, whether the first user has an illness risk; and
in response to determining that the first user has an illness risk, send to the first user an illness risk notification.

2. The system according to claim 1, wherein the processor is configured to:
determine, based on the first criteria and the first data, whether the first user is to be in quarantine; and
in response to determining that the first user is to be in quarantine, send to the first user an illness quarantine notification including time and duration of quarantine.

3. The system according to claim 1, wherein in setting the first criteria, the processor is configured to set a criterion for each of (1) vital information, (2) questionnaire response information, (3) vaccination information, and (4) illness test result information.

4. The system according to claim 1, wherein in setting the first criteria, the processor is configured to set a group of users to which the first criteria are to be applied, wherein the group of users includes the first user.

5. The system according to claim 3, wherein in determining whether the first user has an illness risk, the processor is configured to:
compare each of the information (1) to (4) of the first data with the corresponding criterion; and
determine, based on a result of the comparison, whether the first user has an illness risk.

6. The system according to claim 1, wherein the processor is configured to:
set second criteria for (1) vital information, (2) questionnaire response information, (3) vaccination information, and (4) illness test result information, the second criteria relating to a second illness different from the first illness.

7. The system according to claim 1, wherein the vital information of the first user comprises a measured body temperature of the first user.

8. The system according to claim 1, wherein the questionnaire response information of the first user comprises one or more responses to one or more questions relating to symptoms of the first illness.

9. The system according to claim 1, wherein the vaccination information of the first user comprises information relating to administration of one or more doses of vaccine of the first illness to the first user.

10. The system according to claim 1, wherein the illness test result information of the first user comprises information relating to administration of one or more tests of the first illness to the first user.

11. A method for controlling notifications relating to illness information, the method comprising:
setting, by a processor, first criteria for (1) vital information, (2) questionnaire response information, (3) vaccination information, and (4) illness test result information, the first criteria relating to a first illness;
receiving, by the processor, first data relating to an illness risk of a first user, the first data comprising (1) vital information, (2) questionnaire response information, (3) vaccination information, and (4) illness test result information, of the first user;
determining, by the processor based on the first criteria and the first data, whether the first user has an illness risk; and
in response to determining that the first user has an illness risk, sending, by the processor, to the first user an illness risk notification.

12. The method according to claim 11, further comprising:
determining, based on the first criteria and the first data, whether the first user is to be in quarantine; and
in response to determining that the first user is to be in quarantine, sending to the first user an illness quarantine notification including time and duration of quarantine.

13. The method according to claim 11, wherein setting the first criteria comprises:
setting a criterion for each of (1) vital information, (2) questionnaire response information, (3) vaccination information, and (4) illness test result information.

14. The method according to claim 11, wherein setting the first criteria comprises:
setting a group of users to which the first criteria are to be applied, wherein the group of users includes the first user.

15. The method according to claim 13, wherein determining whether the first user has an illness risk comprises:
comparing each of the information (1) to (4) of the first data with the corresponding criterion; and
determining, based on a result of the comparison, whether the first user has an illness risk.

16. The method according to claim 11, further comprising:
setting second criteria for (1) vital information, (2) questionnaire response information, (3) vaccination information, and (4) illness test result information, the second criteria relating to a second illness different from the first illness.

17. The method according to claim 11, wherein the vital information of the first user comprises a measured body temperature of the first user.

18. The method according to claim 11, wherein the questionnaire response information of the first user comprises one or more responses to one or more questions relating to symptoms of the first illness.

19. The method according to claim 11, wherein the vaccination information of the first user comprises information relating to administration of one or more doses of vaccine of the first illness to the first user.

20. The method according to claim 11, wherein the illness test result information of the first user comprises information relating to administration of one or more tests of the first illness to the first user.
